(19) United States
(12) Patent Application Publication
(10) Pub. No.: US 2004/0258651 A1
(43) Pub. Date: Dec. 23, 2004

(54) Use of alkylguanidine compounds for the treatment and after-treatment of hair

(75) Inventors: Matthias Pascaly, Essen (DE); Nicole Flintrop, Duisburg (DE); Burghard Gruning, Essen (DE); Holger Leidreiter, Hattingen (DE); Peter Lersch, Dinslaken (DE); Peter Muss, Essen (DE); Christian Weitemeyer, Essen (DE)

Correspondence Address: Leopold Presser, Scully, Scott, Murphy & Presser, 400 Garden City Plaza, Garden City, NY, 11530, US

(73) Assignee: GOLDSCHMIDT AG, ESSEN (DE)

(21) Appl. No.: 10/858,933

(22) Filed: Jun. 2, 2004

(30) Foreign Application Priority Data
Jun. 18, 2003 (DE) 103 27 871.0

(51) Int. Cl. A61K 7/09; A61K 7/075; A61K 7/08

(52) U.S. Cl. 424/70.27

(57) ABSTRACT

Hair treatment compositions and hair treatment compositions are provided for improving the combability, handle, disentanglability, volume and luster, which comprises additionally using, as active substances, at least one of the compounds of the general formulae (I) and/or (II).

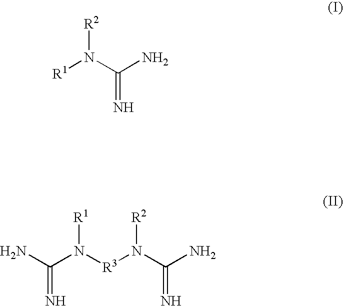

### FIELD OF THE INVENTION

The present invention relates to a personal care product and more particulary to a hair treatment or after-treatment composition that includes an alkylguanidine compound. The present invention is also related to cosmetic formulations, hair shampoos, hair conditioning compositions and leave-in formulations which include at least the composition of the present invention.

### BACKGROUND OF THE INVENTION

Human hair is exposed daily to all sorts of influences. In addition to mechanical stresses due to brushing, combing, putting up or tying back, the hair is also attacked by environmental influences such as, for example, strong UV radiation, cold, wind and water. The physiological status (e.g., the age, and health) of the particular person also influences the damage to the keratinic fibers.

In particular, however, treatment with chemical agents changes the structure and surface properties of the hair. Methods such as, for example, permanent waving, bleaching, dyeing, tinting, smoothing etc, as well as frequent washing with aggressive surfactants, contribute to more or less severe damage being caused to the hair structure. The surface of the hair roughs up, making it difficult to comb and brittle. Thus, for example, during permanent waving both the cortex and the cuticle of the hair is attacked. The disulfide bridges of the cystine are broken open by the reduction step and partly oxidized to cysteic acid in the subsequent oxidation step. These anionic structures on the surface of the hair cause a loss of softness and smoothness in the wet and dry state.

With respect to bleaching, not only the melamine is destroyed, but moreover about 15 to 25% by weight of the disulfide bonds of the cystine are oxidized on mild bleaching. Excessive bleaching can cause up to 45% by weight of the disulfide bonds of the cystine to be oxidized (K. F. de Polo, A Short Textbook of Cosmetology, 2000, Verlag für chemische Industrie, H. Ziolkowsky GmbH).

Thus, disadvantageous mechanical properties for the hair result from the chemical treatments, the frequent washing or the UV irradiation, caused by removal of naturally secreted hair fats and humectants (sebum). Hair thus becomes brittle, dry, lustrless, porous and feels rough. Moreover, thoroughly cleansed hair is usually very difficult to comb, both in the wet and in the dry state, since the individual hairs tend to become tangled and to knot. On drying, the hair does not set in the desired manner. At the same time, the hair remains difficult to comb and additionally also has disadvantageous electrostatic properties.

In this state, the hair is easily accessible to further damage by chemicals, surfactants and environmental influences, whereby hair breakage and splits can occur. Hence, the altogether unsatisfactory state of cleansed or damaged hair necessitates a subsequent treatment by a conditioning formulation in order to improve the undesired properties of the hair.

Conditioning agents are thus used separately from shampoos and are normally rinses, cream emulsions or lotions such as, for example, hair rinses, hair tonics, shampoos, leave-in conditioners, and etc. having at least one cationic component.

For the definition of the terms conditioning agent or conditioner/conditioning agents, a wide basis is found in the literature and is defined by Breuer et al. “Physical chemistry of hair condition”, Cosmetics & Toiletries 94 (1979), 29 and by Edelstein “Hair Conditioners and Conditioning”, Cosmetics & Toiletries 100 (1985), 31 as the effect which occurs after a hair treatment (with formulations comprising these conditioning agents) and improves the properties of the hair with respect to combability, shapeability, volume, handleability and optical properties (Breuer) or combability, disentangleability, luster, volume, strength and shapeability (Edelstein).

In Ullmann's Encyclopedia of Industrial Chemistry (2003), 6th ed., “Hair Preparations, 3.2 Conditioning Agents and Treatments”, hair conditioning agents are defined as follows: Hair conditioning agents prevent, delay or conceal changes in the hair caused by environmental influences and chemical treatment agents. The action is based mainly on a superficial change in the hair, whereby even its luster is positively influenced. Almost all modem shampoos, styling lotions and colorings contain conditioning additives which prevent excessive mechanical damage to the hair. Simple conditioning agents, however, are not suitable for long or chemically treated hair. In this case, special conditioning additives must be additionally used.

Analogous definitions are to be found in the patent literature. Thus, in WO-A-97/09028 it is stated that a conditioning, i.e., that is a general improvement in the state of the hair, can be brought about by additives penetrating into the hair, in particular in damaged areas of the hair, or accumulating on such areas and in this way improving its general state, for example, its combability.

In WO-A-91/04007, the “conditioning effect” is understood as meaning an improvement in the combability of wet and dry hair, in the luster, in the silky, smooth and soft feel, and in the handleability on styling and setting.

Conventional leave-in and rinse-off conditioning agents contain fatty alcohols for adjusting the viscosity and quaternary alkylammonium compounds and silicones for actual conditioning. In this process, the fatty alcohols are prone to deposition on the individual hairs and ultimately cause a decrease in the volume.

The surface of the hair is anionically charged in the untreated state, caused by carboxylate and sulfonic acid groups. Disadvantageous electrostatic properties result from this. For the saturation of the anionic charges, in commercially available haircare compositions, mainly alkylammonium-based cationic surfactants or silicone quats are admixed. On account of coulombic interactions, these compounds accumulate on the oppositely charged hair surface, on which they form a monomolecular layer and thereby hydrophobize the hair. On account of the steric hindrance, alkylammonium compounds, however, show only a weak hold on hair caused by coulombic interactions.

There is therefore a need for ingredients which can be employed in a versatile manner for hair treatment agents and hair aftertreatment agents, which improve the combability, the handle and the luster of the hair for a long time afterward and to an increased extent, and suppress or eliminate the undesired concomitant symptoms of hair treatments.

Preferably, such an active ingredient, even in small use concentrations, should cause a marked effect, be nontoxic, preferably of natural origin or naturally identical, be very well tolerated by the hair and the scalp, have a high compatibility with other ingredients and be able to be incorporated into hair treatment compositions and hair aftertreatment compositions without problems. It is particularly desirable if this ingredient can additionally exert the function of certain components which have been employed hitherto for hair treatment, such as, for example, that of a conditioning agent.

### SUMMARY OF THE INVENTION

It is an object of the present invention to make available an active ingredient that is able to improve both the sensory properties of hair in the dry and wet state, and to eliminate the damage to the hair caused by washing, chemical treatments or exogenous factors, and thus accordingly has a broader spectrum of action than conditioning agents.

It has now surprisingly been found that not only simple alkyl-guanidine compounds and their derivatives but also their acid conjugates in preparations for the treatment and after-treatment of the hair fulfill all these desired criteria and markedly surpass commercially available compounds in performance.

Specifically, the present invention provides hair treatment compositions and hair after-treatment compositions that include, as conditioning agents, 0.05 to 10.0% by weight of at least of an alkylguanidine compound of the general formulae (I) and/or (II)

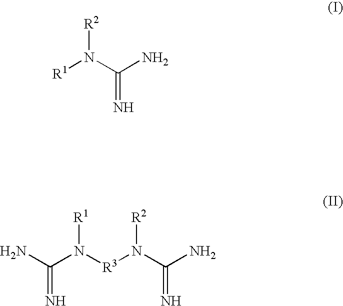

and/or their salts or hydrates, in which

R¹, R² a) independently of one another are H, an optionally branched hydrocarbon radical optionally containing double bonds, hydroxy-alkyl, alkyloxy, carboxyalkyl radicals, having 2 to 30 C atoms, preferably 4 to 22, in particular 6, 8 or 16 to 22, C atoms, or

b) alicyclic, heterocyclic compounds optionally containing multiple bonds, which can have a ring size of 3 to 10 atoms, preferably of 4 to 6 atoms, which can carrey further, saturated or unsaturated hydrocarbon substituents having 1 to 30 C atoms, preferably 4 to 22 C atoms, or

c) alkylamidoalkylene or alkyl ester alkylene radicals, which can further contain structural elements mentioned under a) and b), with the proviso that in compounds as shown in formula (I) where R¹=alkylamidoalkylene, R² is not hydrogen,

and the structural elements a), b), c) can be combined among one another and with one another,

R³ is alkylene, an optionally branched hydrocarbon radical having 1 to 30 C atoms, preferably 4 to 22 C atoms, optionally containing double bonds or alicyclic or heterocyclic components, which is saturated, unsaturated or aromatic, having a ring size of 3 to 10 atoms, preferably having a ring size of 4 to 6 atoms, or in which R¹ and R² in the element —N(R¹)—R³—(R²)N— can form a 5- to 8-membered ring.

The present invention also relates to the use of alkylguanidine compounds of the general formulae (I) and/or (II) wherein at least one ofihe radicals R1, R² is C₈- or a C₁₆₋₂₂-alkyl radical.

The present invention also relates to the use of alkylguanidine compounds of the general formulae (I) and/or (II) wherein the radicals R¹, R² are C₁₂-alkyl radicals.

The present invention is also directed to the use of alkylguanidine compounds of the general formulae (I) and/or (II) wherein the radicals R¹, R² are identical or different alkylamidoalkylene or alkyl ester alkylene radicals.

The present invention is further directed to the use of alkylguanidine compounds of the general formulae (I) and/or (II) wherein R³ is hydrocarbon radicals, preferably alkylene radicals having 4 to 18, preferably 6 to 12, C atoms and R¹ and R² have the meaning indicated above and are preferably hydrogen at least once.

### DETAILED DESCRIPTION OF THE INVENTION

The alkylguanidines used or additionally used according to the present invention, in particular the straight-chain alkylguanidines, have both good stability and good formulability, produce a marked action even in the low use concentrations, are not toxic, of natural origin or naturally identical, are very well tolerated by the hair and the scalp, have a high compatibility with other ingredients and can be incorporated into hair treatment compositions without problems. Additionally, they can also have a slight antimicrobial action.

The preparation of alkylguanidines is described in DE-C-506 282 (Schering). In the process, alkylamines are guanidylated in an alcoholic solution using cyanamide in the presence of a protonic acid. The products are thus obtained as crystalline salts.

The fatty amines employed for the preparation of the alkylguanidines additionally used according to the present invention are prepared according to known processes by reaction of fatty acids with NH₃ in the presence of catalysts to give the nitrile and subsequent hydrogenation to give the primary amine.

Additionally used amines are obtained from individual fatty acids or mixtures of fatty acids such as caprylic acid, capric acid, 2-ehylhexanoic acid, lauric acid, mylistic acid, palmitic acid, palmitoleic acid, isostearic acid, stearic acid, hydroxystearic acid (ricinoleic acid), di-hydroxystearic acid, oleic acid, linoleic acid, petroselinic acid, elaidic acid, arachic acid, behenic acid and erucic acid, gadoleic acid, and the technical mixtures obtained in the pressure cleavage of natural fats and oils, such as oleic acid, linoleic acid, linolenic acid and in particular rapeseed oil fatty acid, soybean oil fatty acid, sunflower oil fatty acid, tall oil fatty acid, coconut oil. In principle, all fatty acids having a similar chain distribution are suitable. In some embodiments of the present invention, the content of unsaturated components of these fatty acids or fatty acid esters, can be adjusted to a desired iodine number by means of the known catalytic hydrogenation processes or achieved by mixing completely hydrogenated fat components with nonhydrogeneted fat components.

Preferably, partially hardened C₈₋₁₈-coconut or palm fatty acids, rapeseed oil fatty acids, sunflower oil fatty acids, soybean oil fatty acids and tall oil fatty acids, having iodine numbers in the range from about 80 to 150 and in particular technical C₈₋₁₈-coconut fatty acids are employed, where, if appropriate, a choice of citrans isomers such as elaidic acid-rich C_{16/18}-fatty acid cuts can be of advantage. The foregoing are commercially available products and are supplied by various companies under their respective trade names.

In the last ten years, a few patent applications have already been published in which the use of alkylguanidines in haircare compositions is described. Thus, the not very specific application as a cationic surfactant is described in JP-A-06-263621 (Lion) and in JP-A-11-035424 (Kao). Just as nonspecific is the described use of the compounds according to JP-A-06-183931 (Lion) in bairstling formulations and according to JP-A-10-17442 (Kao) in chemical hair treatment compositions.

The use of alkylguanidine derivatives is thus restricted, according to known prior art to the utilization of the surfactant properties for use as detergent substances. Hitherto nothing is to be found about the surprising good properties of this class of compound for the conditioning of hair.

Alkylguanidines of the general formula (I) and/or (II) and/or their salts and/or their hydrates have proven particularly suitable within the meaning of the present invention in which the radicals R¹ and/or R² are a C₆-, C₈- or a C₁₆₋₂₂-alyl radical at least once.

In principle, all cosmetically innocuous inorganic or organic mono- or polybasic acids are suitable for salt formulation, such as, for example, formic acid, acetic acid, propionic acid, heptanoic acid, caprylic acid, nonanoic acid, capric acid, undecanoic acid, lauric acid, myristic acid, pannitic acid, stearic acid, arachic acid, behenic acid, cyclopentane arboxylic acid, cyclohexanecrboxylic acid, acryic acid, methacylic acid, vinylacetic acid, crotonic acid, 2-/3-/4-pentenoic acid, 2-/3-/4/5-hexenoic acid, lauroleic acid, myristoleic acid, pahittoleic acid, oleic acid, gadoleic acid, sorbic acid, linoleic acid, linolenic acid, pivalic acid, ethoxyacetic acid, phenylacetic acid, lactic acid, 2-ethylhexanoic acid, oxalic acid, glycolic acid, maic acid, malonic acid, succinic acid, tararic acid, glutaric acid, citric acid, adipic acid, pimelic acid, suberic acid, azelaic acid, sebacic acid, benzoic acid, o-/m-/ptoluic acid, phenylacetic acid salicylic acid, 3-/4-hydroxybenzoic acid, phthalic acids, or their completely or partly hydrogenated derivatives such as hexahydro- or tetrahydrophthalic acid, carbonic acid, phosphoric acid, hydrochloric acid, sulfuric acid and their mixtures are very highly suitable, in particular lactic acid, tartarc acid, acetic acid and hydrochloric acid. Here, it is also possible within the meaning of the present invention to use both suitable guanidine derivatives in mixtures with one another and mixed salts.

Cosmetic preparations according to the present invention for the after-treatment, shaping and care of the hair are especially understood as meaning those hair treatment compositions which are used after a chemical treatment of the hair (chemical hair after-treatment compositions and hair treatment compositions by means of which the hair structure is adversely influenced) where an improvement in the combability, handle, disentangleability, volume and luster of hair is obtained by the addition of alkylguanidine compounds.

Alklyguanidines can generally be present in the present invention in a concentration of 0.01 to 10.0% by weight, preferably in a concentration of 0.1 to 5.0% by weight, in particular in a concentration of 0.1 to 2.0% by weight.

The hair after-treatment compositions are, for example, hair rinses, hair tonics, reviving compositions, leave-in conditioners, hair shampoos, two-in-one shampoos, setting formulations such as foam setting agents, hair spray or hair dryer lotions, hair lotions, hair tip fluids. They can be present as a get emulsion, solution, aerosol spray or foam, nonaerosol spray or foam.

The cosmetic preparations according to the present invention for the treatment of the hair after a chemical treatment have a pH of 3 to 7 and therefore preferably contain a water-soluble acid or a buffer mixture suitable therefor, which stabilizes this pH.

The cosmetic preparations according to the present invention for the treatment of the hair after a chemical treatment can, in addition to alkylguanidine compounds, contain further components which are advantageous and/or customary for the particular application purpose.

Thus, for example, shampoos can contain, for example, 3 to 30% by weight of foaming anionic, zwitterionic, ampholytic and nonionic surfactants. Hair rinses and hair tonics contain 0 to 10% by weight, preferably 0.5 to 5% by weight, of emulsifiers, 0 to 10% by weight, preferably 0.5 to 5% by weight, of consistency-imparting agents and 0 to 20% by weight of cosmetic oils of plant and synthetic origin, emollients, vitamin preparations and proteins. Shampoos, hair rinses, hair tonics and reviving agents moreover preferably contain 0 to 8% by weight, preferably 0.1 to 5% by weight, of cationic surfactants and water-soluble polymers having quaternary ammonium groups for lowering the static chargeability and for improving combability, handle and luser.

The cationic surfactants are as a rule

quaternary ammonium compounds, such as, for example, alkyltrimethylammonium salts, dialkyldimethylammonium salts, trialkylmethylammonium salts and imidazolinium compounds. The long alkyl chains consist of a carbon chain having 10 to 22 C atoms, the counterions to the quaternary nitrogen are, for example, halides, sulfate, acetate, lactate, glycolate, nitrate or phosphate. Products are found on the market under the name Varssoft® 300,432 CG, 442-100 P, BT 85 from Goldscbmidt Rewo, Dehyquart® A from Henkel;

esterquats, such as are marketed under the name Dehyquart® F75 by Henkel or Armocare® VGH-70 by Akzo;

alkylamidoquats, such as are commercially available, for example, under the name Varisoft® PATC and RTM 50 from Goldschmidt Rewo.

The water-soluble polymers having quaternary ammonium groups are, for example,

cationic cellulose derivatives, such as are commercially obtainable under the name Celquat® H 100 and L 200 from National Starch or Polymer JR® 400 from Amerchol,

polymeric dimethyldiallylamlonium salts and their copolymers with esters and amides of acrylic acid and methacrylic acid The products obtaiable commercially under the name Merquat® 100 or Merquat® 550 from Calgon are examples of such cationic polymers,

copolymers of vinylpyrohdone with quaternized derivatives of dialkyl aminoacrylate and mecrylate. Such compounds are commercially obtainable under the name Gafquat® 735 and Gafquat®744 from ISP, vinylpyrolidone/vinylimidazolium methochloride copolymers, such as are supplied under the name Luviquat® FC 370, FC 550, FC 905 and HM-552 from BASF,

quaternized polyvinyl alcohol,

quaternized protein hydrolyzates of animal or vegetable origin based on keratin, collagen, elasin, wheat, rice, soybeans, milk silk corn. Such products are marketed, for example, under the name Croquat® Wheat and Silk from Croda, Promoisg W-32CAQ, Silk CAQ, WG CAQ from Seiwa Kasei or QuatColl® CDMA from Brooks,

guar hydroxypropyltrimethylammonium chloride,

aminofunctional polydimethylsiloxanes or hydroxylamino-modified silicones, such as the commercial products ABIL® Quat 3272 and ABIL® Quat 3474 from Goldschmidt, Dow Corning® 929 emulsion, Dow Corning® 939 from Dow Corning.

Setting agent formulations and other hair styling preparations customarily contain 0.1 to 5% by weight of film-forming polymers soluble in aqueous or aqueous-alcoholic media, optionally together with cationic surfactants or cationic polymers. Examples of film-forming agents are homo-polymers of vinylpynolidone, homopolymers of N-vinyl-formamide, copolymers of vinylpynolidone and vinyl acetate, terpolymers of vinylpyrrolidone, vinyl acetate and vinyl propionate, polyacrylamides, polyvinyl alcohols, high molecular weight polyethylene glycol or high molecular weight copolymers of ethylene glycol with propylene glycol, chitosan. These products are found on the market under the name Luviskol® K30, K60, K80, VA37E from BASF or PVPNA E335 and PVP K30 from ISP.

Typical basic recipes for the reepective applications are known prior art and are contained, for example, in the brochures of the manufact of the respective raw materials and active mgredients. These existig formulations can as a rule be adopted unchanged. If required for adaptation and optimization, the desired modifications, however, can be performed without complications by means of simple experiments.

A typical formulation for a hair rinse/hair tonic contains, for example:

a) 0.1 to 2% by weight of at least one of the compounds of the general formula (I) and/or (II),

b) 0.1 to 5% by weight of emulsifier,

c) 0.1 to 5% by weight of consistency-imparting agent,

d) 0.1 to 5% by weight of cationic surfactants and/or water-soluble polymers having quaternary ammonium groups,

e) 0 to 10% by weight of other cosmetic active compounds, preservatives, and customary additives and excipients, and

f) water to 100% by weight

A typical formulation for a hair shampoo contains, for example:

a) 0.1 to 2% by weight of at least one of the compounds of the general formula (I) and/or (I),

b) 3 to 30% by weight of foaming anionic, amphoteric, ampholytic or nonionic surfactants,

c) 0.1 to 5% by weight of cationic surfactants and/or water-soluble polymers having quaternary ammonium groups,

d) 0.1 to 6.0% by weight of thickener,

e) 0 to 10% by weight of other cosmetic active compounds, opacifying agents, solvents, and customary additives and excipients, and

f) water to 100% by weight

The preparations for chemical hair treatment, in which the compounds of the general formula (I) and/or (II) can additionally be used with success according to the present invention, are compositions for the permanent shaping of the hair such as permanent waving agents and fixing agents or hair-smoothing agents, color-changing agents such as bleaching agents, oxidation coloring agents and tinting agents and shampoos based on direct dyes.

Such preparations can contain in the case of a permanent wave solution, for example, 1 to 10% by weight of thioglycolic acid, thioglycolic acid salts or esters, permanent wave setting agents or bleaching agents, preferably 2 to 10% by weight of oxidants, such as, for example, potassium bromate, sodium bromate or hydrogen peroxide. Hair-smoothing agents are based on the use of strong bases or on reducing agents such as, for example, thioglycolic acid salts. Hair-coloring agents contain direct hair-coloring agents or oxidation dye precursors.

Finally, the preparations according to the present invention can also contain cosmetic excipients and additives which are customary in such preparations. Such excipients are, for example, solubiliiers such as ethanol isopropanol, ethylene glycol, propylene glycol, glycerol and diethylene glycol, complexing agents such as EDTA, NTA, β-alaninediacetic acid and phosphoric acid, preservatives, antioxidants, fragrances, colorants for coloring the cosmetic preparation, opacifying agents such as latex, styrene/PVP and styrene-acrylamide copolymers, pearl luster agents such as ethylene glycol mono- and disteate and PEG-3 distearate, pigments, light-screens, thickening agents or propellants.

The alkylguanidine compounds can also be combined in the cosmetic preparations according to the present invention with other hair cosmetic active ingredients, such as, for example, ceramides, pseudoceramides, protein hydrolyzates of vegetable or animal origin based on keratin, collagen, elastin, wheat, rice, soybeans, milk, silk corn, but also amino acids and antidandruff active ingredients such as piroctone olamine, zinc omadine and climbazole, sebostatics, vitamins, panthenol, pyrolidon carboxylic acid, bisabolol, or plant extracts.

The hair treatment compositions according to the present invention are prepared in the customary manner, the alkylguanidine compounds being dissolved both in the aqueous and in the oil phase. The pH is preferably finally adjusted by addition of the acid and/or of the buffer mixture intended therefor.

Some preparation and use examples and recipes are given in the following text. These illustrate the subject of the present invention and do not restrict this.

### PREPARATION EXAMPLES

Preparation of octylguanidinium acetate and C₁₆₋₂₂-alkyl-guanidinium acetate:

30.7 g of octylamine or 70.8 g of C₁₆₋₂₂-alkylamine (0.2375 mol) were dissolved in a 30 ml of n-butanol at elevated temperature with stirring. During the warming phase, 14.2 g of acetic acid (0.2365 mol) was added. After reaching the reaction temperature of 90° C., a solution of 10.0 g of cyanamide (0.2379 mol) in butanol (about 60 mol) was added dropwise over a period of 3 h and the batch was stirred further at 90° C. for 3 h. After cooling, the solvent was stripped off on a rotary evaporator under reduced pressure. The product was suspended using acetone, filtered off and washed with diethyl ether. The final product was present as a crystalline colorless powder.

Octylguanidinium acetate: ¹³C-NMR, 100 Mlz, CD₃OD, 25° C.: δ=181.0 (1C, COOH_{AcoH}), 159.3 (1C, C_{guanidinium gr.}), 42.9 (1C, CH₂), 33.4 (1C, CH₂), 30.8 (1C, CH₂), 30.4 (1C, CH₂), 28.2 (1C, CH₂), 24.8 (1C, CH₂), 24.1 (1C, CH_{3,AcOH}), 14.9 (1C, CH₃); MALDI-TOF[m/z]: 172.2 (M+H⁺).

C₁₆₋₂₂-Alkylguanidinium acetate: ¹³C-NMR, 100 MW, CD₃OD, 50° C.: δ=178.5 (1C, COOH_{AcOH}), 157.1 (1C, C_{guanidine gr.}), 40.6 (1C, CH₂), 31.2 (1C, CH₂), 28.9 (1C, CH₂), 28.5 (1C, CH₂), 28.5 (1C, CH₂), 28.1 (1C, CH₂), 25.9 (1C, CH₂), 22.5 (1C, CH₂), 21.8 (1C, CH_{3,AcOH}), 12.6 (1C, CH₃); MALDI-TOF[m/z]: 284.2 (C₁₆-guanidine+H⁺), 312,2 (C₁₈-guanidine+H⁺), 340.2 (C₂₀-guanidineH⁺), 368.3 (C₂₂-guanidine+H⁺).

Preparation of Octylguanidinium Chloride:

15.4 g of octylarine (0.1194 mol) and 20 ml of n-butanol were initially introduced and warmed to 90° C. with stirring. During the warming phase, 1.0 g of hydrochloric acid (37%; 0.1137 mol) was added. After reaching the reaction temperature of 90° C., over a period of 3 h a solution of 5.0 g of cyanamide (0.1308 mol) in butanol (about 60 ml) was added dropwise and the batch was stirredd further at 90° C. for 4 h. After cooling, the solvent was stripped off on a rotary evaporator under reduced pressure. The product was dissolved in H₂O and separated off using NaCl solution. Drying of the product was carried out by addition of ethanol and subsequent distillation. The final product was a highly viscous colorless liquid.

¹³C-NMR, 100 MHz, CD₃OD, 25° C.: δ=158.6 (1C, C_{guanidinium gr.}), 42.9 (1C, CH₂), 42.8 (1C, CH₂), 33.0 (1C, CH₂), 30.4 (1C, CH₂), 29.9 (1C, CH₂), 27.8 (1C, CH₂), 23.8 (1C, CH₂), 14.8 (1C, CH₃); MALDI-TOF[m/z]: 172.2 (M+H⁺), 192.9 (M+Na⁺).

Preparation of Octylguanidiniumn Lactate:

15.4 g of octylamine (0.1194 mol) and 20 ml of n-butanol were introduced into a multinecked round-bottomed flask, and warmed to 90° C. with stirring. During the warming phase, 10.2 g of lactic acid (0.1137 mol) was added. After reaching the reaction tens of 90° C., over a period of 3 h a solution of 5.0 g of cyanamide (0.1194 mol) in butanol (about 60 ml) was added dropwise and the batch was stirred further at 90° C. for 3 h. After cooling, the solvent was stripped off on a rotary evaporator under reduced pressure. The product was suspended using acetone, filtered off and washed with diethyl ether. The final product was present as a crystlline colorless powder.

¹³C-NMR, 100 MHz, CD₃OD, 20° C.: δ=182.4 (1C, COOH_{lactic acid}), 158.7 (1C, C_{guanidinium gr.}), 69.6 (1C, COH_{lactic acid}), 42.5 (1C, CH₂), 33.0 (1C, CH₂), 30.34 (1C, CH₂), 30.0 (1C, CH₂), 27.8 (1C, CH₂), 23.7 (1C, CH₂), 21.7 (1C, CH_{3,latic acid.}), 14.5 (CH₃), MALDI-TOF[m/z]: 172.1 (M+H⁺).

Preparation of hexane-1,6-diguanidinium acetate:

36.27 g (0.312 mol) of 1,6 diaminohexane, 37.49 g (0.624 mol) of acetic acid and 180 ml of n-butanol were introduced and warmed to 90° C. The cyanamide was dissolved in 80 ml of 1-butanol and continuously added dropwise at 90° C. over the course of 3 hours. After completion of the addition, the mixture was allowed to subsequently react at 90° C. for 4 hours. The solvent was stripped off on the rotary evaporator. The residue was then recrystallized from water, washed twice with 50 ml of acetone each time and the product was dried at 60° C. in vacuo.

The final product was obtained as a crystalline pale beige powder.

¹³C-NMR, 100 MHz, D₂O, 20° C.: δ=181.1 (2C, COOH_{AcOH}), 156.6 (2C, C_{guanidinium gr.}), 40.9 (2C, CH₂), 27.6 (2C, CH₂), 25.3 (2C, CH₂), 23.2 (2C, CH_{3,AcOH}); MALDI-TOF[m/z]: 201.2 (M+H⁺).

Preparation of N,N-dibutylguanidinium acetate:

55.86 g (0.432 mol) of dibutylamine, 25.96 g (0.432 mol) of acetic acid and 100 ml of 1-butanol were introduced and warmed to 90° C. The cyanamide was dissolved in 60 ml of n-butanol and continuously added dropwise at 90° C. over the course of 3 hours. After completion of the addition, the mixture was allowed to subsequently react at 90° C. for 4 hours. At 76° C., the precipitated salt was filtered off and washed twice with 50 ml of acetone each tine. The product was dried at 60° C. in vacuo on the rotary evaporator, and the final product was obtained as a colorless crystaline substance.

¹³C-NMR, 100 MHz, ethanol-d₆, 20° C.: δ=178.8 (1C, COOHH_{AcOH}), 155.9 (1C, C_{guanidinium gr.}), 48.1 (2C, CH₂), 29.0 (2C, CH₂), 23.3 (1C, CH_{3,AcOH}), 19.2 (2C, CH₂), 12.9 (2C, CH₂); MALDI-TOF[m/z]: 172.2 (M+H⁺).

### APPLICATION EXAMPLES

For the application technology assessment, hair tresses which were used for sensory tests were predamaged in a standardized manner by a permanent wave treatment and a bleaching treatment For this, customary hairdresser's products were used.

Materials:

permanent wave liquid (e.g. “ondi”, Wella)

setting agent (e.g. “neutrafix”, Wella)

bleaching powder (e.g. “blondor special”, Wella)

H₂O₂ (e.g. “Welloxyd 9%”, Wella)

Shampoo without a care component (e.g. sodium lauryl ether sulfate (12% WAS), NaCl thickened)

beaker

hair-coloring brush

The treatment was carried out in the following sequence:

1. Permanent Wave Treatment:

The hair tresses were moistened with the permanent wave liquid (weight ratio of hair: liquid=1:2). After a time of action of 15 min at room temperature in a covered beaker, the permanent wave liquid was carefully rinsed out for 2 min. Subsequently, the strands of hair were gently pressed dry using a towel.

The setting agent (ratio hair: liquid=1:2) had a time of action of 10 min at room temperature. Subsequently, the setting agent was carefully rinsed out for 2 min.

The hair was then dried overnight at room Lathe.

2. Bleaching Treatment

The bleaching powder and the H₂O₂ were processed to give a paste (weight ratio of powder H₂O₂=2:3). The paste was then immediately applied to the permanently waved hair using a brush. The time of action was 30 min at room temperature. Subsequently, the bleaching paste was rinsed out under running water for 2 min. The hair was then washed with a shampoo without conditioner for 1 min (amount of shampoo: 0.5 ml/hair tress) and then rinsed out for 1 min.

Before the predamaged strands of hair were used for sensory function tests, they were dried overnight (12 h) at room temperature.

Test Recipe:

The test recipe contained the conditioning products according to the present invention or the combination of products according to the present invention in a cosmetically acceptable form. As test recipes, it was possible to use hair shampoos, hair rinses, hair tonics (hair balm), leave-in conditioners and other formulations. For comparison, comparison samples from the prior art were tested. These comparison test recipes contained products or combination of products which correspond to the prior art.

Standardized treatment of predamaged strands of hair with conditioning samples:

The strands of hair predamaged as described above were treated with the conditioning rinse described above as follows: The strands of hair were wetted under flowing, warm water. The excess water was lightly pressed out by hand, then the rinse was applied and gently incorporated into the hair (1 ml/strand of hair (2 g)). After a residence time of 1 min, the strand of hair was rinsed for 1 min. Before the sensory assessment the hair was dried in air at 50% atmospheric humidity and 25° C. for at least 12 h.

Assessment Criteria:

The sensory assessment was carried out according to marks which were awarded on a scale of 1 to 5, where 1 was the poorest and 5 the best assessment.

TABLE 1
Wet combability:

| | | |
|---|---|---|
| 5 | Coarse type of teeth | No knots, the hair can be readily disentangled |
| | [of the comb] | |
| | fine type of teeth | Very easy combing through, no resistance |
| | | noticeable |
| 4 | coarse type of teeth | Isolated knots. The hair can be readily |
| | | disentangled |
| | fine type of teeth | Easy combing through, low resistance |
| | | noticeable |
| 3 | coarse type of teeth | A few knots, low resistance |
| | fine type of teeth | Some resistance noticeable, which declines |
| | | after combing several times |
| 2 | coarse type of teeth | A few knots, marked resistance |
| | fine type of teeth | Marked resistance, which does not decline after |
| | | combing several times |
| 1 | coarse type of teeth | Many knots, strong resistance |
| | fine type of teeth | Very strong resistance, sometimes the hair |
| | | cannot be combed through |

TABLE 2
Wet handle:

| | |
|---|---|
| 5 | very smooth, soft but despite this beautifully firm, easy to handle, |
| | not greasy/sticky (no residues noticeable) |
| 4 | smooth and soft and/or only small residues noticeable |
| 3 | smooth, somewhat hard and/or some residues noticeable |
| 2 | hard and/or noticeably greasy, waxy residues |
| 1 | very hard, rough, dull and/or extremely greasy, sticky (markedly |
| | noticeably greasy, waxy residues noticeable) |

TABLE 3
Dry combability:

| | | |
|---|---|---|
| 5 | coarse type of teeth | no knots, the hair can be readily disentangled |
| | Fine type of teeth | very easy combing through, no resistance |
| | | noticeable, the hair does not become charged |
| 4 | coarse type of teeth | isolated knots, the hair can be readily |
| | | disentangled |
| | Fine type of teeth | easy combing through, low resistance |
| | | noticeable, hair becomes minimally charged |
| 3 | coarse type of teeth | some knots, low resistance |
| | Fine type of teeth | some resistance noticeable, which declines |
| | | after combing several times, hair does |
| | | not become very charged |
| 2 | coarse type of teeth | few knots, marked resistance |
| | Fine type of teeth | marked resistance, which does not decline |
| | | after combing several times, hair becomes |
| | | charged |
| 1 | coarse type of teeth | many knots, strong |
| | | resistance |
| | fine type of teeth | very strong resistance, sometimes the hair |
| | | cannot be combed through, hair becomes |
| | | markedly charged |

TABLE 4
Dry handle:

| | |
|---|---|
| 5 | very smooth, soft but despite this firm, full, easy to |
| | handle |
| 4 | smooth and soft |
| 3 | smooth, slightly hard and/or slightly dull (residues) |
| 2 | hard, somewhat dull |
| 1 | rough, hard, dry, dull (residues) |

TABLE 5
Dry appearance:

| | |
|---|---|
| 5 | Extremely shiny |
| 4 | shiny |
| 3 | somewhat shiny |
| 2 | not very shiny, slightly dull |
| 1 | dull, no luster |

In order to assess the volume, the strand of hair was lightly shaken by holding it at the position of the adhesive.

TABLE 6
Assessment criteria of the hair volume:

| | |
|---|---|
| 5 | relatively loose, billowing case, Ø in the tip area rel. large |
| 4 to 2 | Intermediate stages |
| 1 | hair hangs heavily, Ø below the bunching similar to the tip area |

For the determination of the electrostatic behavior of the strand of hair, a “fly-away test” was carried out. In this, the transfer of electrostatic charge to a strand of hair was determined by combing. The spreading open of the strands of hair by electrostatic repulsion of the hairs by one another was measured. For comparison, this measurement was indicated for each conditioning agent relative to the measurement of the control sample (reduction of the spreading open compared with the control sample).

For the indication of a total assessment of the conditioning properties, a mean value was formed from the individual assessments of the disentanglability, wet combability, wet handle, dry combability, dry handle, appearance and volume.

Test Results:

TABLE 7
Formulations of simple hair rinses used with and without
alkylguanidinium compounds according to the present invention:

| | | Hair rinse | Hair rinse |
|---|---|---|---|
| | | Type B | Type C |
| | Hair rinse | Not according to | |
| | Type A | the present invention | |
| Alkylguanidinium-compound | 2.0 | — | — |
| [% by weight] | | | |
| Conditioning agent of the | — | 2.0 | — |
| prior art [% by weight] | | | |
| Ceteareth-25 | 0.5 | 0.5 | 0.5 |
| [% by weight] | | | |
| Cetyl alcohol | 2.0 | 2.0 | 2.0 |
| [% by weight] | | | |
| HCl | to pH = 5 | to pH = 5 | to pH = 5 |
| Water [% by weight] | to 100 | to 100 | to 100 |

In the following tables (Table 8a and 8b), the results of the sensory assessment of the treatment of the stands of hair was carried out as described above using compounds according to the present invention or comparison examples (formulations according to Table 7) are summarized.

TABLE 8a
Results of the sensory assessment after treatment of strands of hair with formulations
according to Table 7

| Test formulation | | | | | | | | |
|---|---|---|---|---|---|---|---|---|
| Compounds | | | | | | | | |
| according to formula | Sensory assessments | | | | | | | |
| (I) where | Disen- | Comb- | | Comb- | | | | Overall |
| R¹ = H present as the | tanglement | ability | Handle | ability | Handle | | | assessment |
| acetic acid salt | wet | wet | wet | dry | dry | Luster | Volume | (mean value) |
| HSP A where R² = C₄ | 4.0 | 3.8 | 4.7 | 4.7 | 5.0 | 4.3 | 3.6 | 4.3 |
| HSP A where R² = C₁₆ | 4.7 | 4.0 | 4.7 | 4.7 | 4.7 | 4.0 | 4.4 | 4.5 |
| HSP A where R² = C₁₈ | 3.8 | 3.8 | 4.2 | 4.2 | 4.5 | 3.4 | 4.5 | 4.1 |
| HSP A where R² = C₂₂ | 4.5 | 4.2 | 4.8 | 4.2 | 4.5 | 4.4 | 5.0 | 4.5 |
| HSP B | 3.7 | 3.7 | 4.0 | 4.5 | 4.2 | 3.3 | 4.6 | 4.0 |
| comparison recipe | | | | | | | | |
| with cetrimonium | | | | | | | | |
| chloride | | | | | | | | |
| (industry standard) | | | | | | | | |
| HSP C | 1.3 | 1.7 | 1.7 | 3.0 | 3.0 | 3.7 | 4.0 | 2.6 |
| comparison recipe | | | | | | | | |
| (control basis) | | | | | | | | |

HSP: hair rinse

It was seen that the compounds according to the present invention showed good conditioning properties, which in some cases were markedly better than those of the industry standard cetrimonium chloride.

TABLE 8b
Results of the sensory assessment after treatment of strands of hair with formulations
according to Table 7

| Test | | | | | | | | |
|---|---|---|---|---|---|---|---|---|
| formulation | | | | | | | | |
| according to | | | | | | | | |
| Tab. 7 | | | | | | | | |
| with | Sensory assessments | | | | | | | |
| compounds | Disen- | Comb- | | Comb- | | | | Overall |
| according to | tanglement | ability | Handle | ability | Handle | | | assessment |
| formula (I) | wet | wet | wet | dry | dry | Luster | Volume | (mean value) |
| HSP A where | 4.0 | 3.8 | 4.7 | 4.7 | 5.0 | 4.3 | 3.6 | 4.3 |
| R¹ = H; | | | | | | | | |
| R² = C₆; lactate | | | | | | | | |
| HSP A where | 4.3 | 4.0 | 4.7 | 4.7 | 4.5 | 4.0 | 3.6 | 4.3 |
| R¹ = H; | | | | | | | | |
| R² = C₁₂; | | | | | | | | |
| lactate | | | | | | | | |
| HSP A where | 4.8 | 4.3 | 4.7 | 4.7 | 4.3 | 4.0 | 4.4 | 4.5 |
| R¹ = H; | | | | | | | | |
| R² = oleyl; | | | | | | | | |
| acetate | | | | | | | | |
| HSP A where R¹ = H; | 4.7 | 4.3 | 4.5 | 4.7 | 4.5 | 4.3 | 4.0 | 4.4 |
| R² = coconut; acetate | | | | | | | | |
| HSP A where R¹ = H; | 2.3 | 2.0 | 2.3 | 2.0 | 3.0 | 4.7 | 4.3 | 2.9 |
| R² = benzyl; acetate | | | | | | | | |
| HSP A where R¹ = C₄; R² = C₄; | 2.7 | 2.3 | 2.0 | 2.7 | 2.3 | 3.0 | 3.7 | 2.7 |
| acetate | | | | | | | | |
| HSP A where R¹ = C₈; R² = C₈; | 4.3 | 4.3 | 4.3 | 4.7 | 4.3 | 4.0 | 4.0 | 4.3 |
| acetate | | | | | | | | |
| HSP A where R¹ = H; | 4.3 | 4.7 | 4.3 | 4.0 | 4.3 | 3.8 | 4.7 | 4.2 |
| R² = H(OCH₂CH₂)₁₄; acetate | | | | | | | | |
| HSP A where R¹ = CH₂CH₂OH; | 4.7 | 4.7 | 4.0 | 4.3 | 4.3 | 4.3 | 4.0 | 4.4 |
| R² = palmoylethyl-amide; acetate | | | | | | | | |
| HSP A where R¹ = H; R² = | 2.3 | 2.3 | 2.3 | 2.5 | 2.3 | 3.0 | 4.0 | 2.7 |
| 6-caproic acid butylamide; | | | | | | | | |
| phosphate | | | | | | | | |
| HSP A where R¹ = H; R² = | 5.0 | 4.7 | 4.3 | 4.3 | 4.3 | 3.7 | 3.3 | 4.2 |
| alkyldimethicone; acetate | | | | | | | | |
| HSP A where R¹ + R² + N = | 1.3 | 1.7 | 1.7 | 3.0 | 3.0 | 3.7 | 4.0 | 2.6 |
| (—(CH₂)₂—O—(CH₂)₂—); | | | | | | | | |
| acetate | | | | | | | | |
| HSP with compounds | | | | | | | | |
| according to formula (II): | | | | | | | | |
| HSP A where R¹, R² = H; | 4.7 | 4.3 | 4.3 | 4.3 | 4.0 | 4.3 | 4.0 | 4.3 |
| R³ = C₆; acetate | | | | | | | | |
| HSP A where R¹, R² = H; | 4.3 | 4.3 | 4.7 | 4.3 | 4.5 | 4.0 | 3.6 | 4.3 |
| R³ = C₁₂; acetate | | | | | | | | |
| Comparison recipes | | | | | | | | |
| HSP B with | 3.7 | 3.7 | 4.0 | 4.5 | 4.2 | 3.3 | 4.6 | 4.0 |
| cetrimonium | | | | | | | | |
| chloride | | | | | | | | |
| (industry standard) | | | | | | | | |
| HSP B with arginine | 1.3 | 1.7 | 1.7 | 3.0 | 3.0 | 3.7 | 4.0 | 2.6 |
| HSP C | 1.3 | 1.7 | 1.7 | 3.0 | 3.0 | 3.7 | 4.0 | 2.6 |
| (control basis) | | | | | | | | |

HSP: hair rinse

It was seen that the compounds according to the present invention showed good conditioning properties, which in some cases were markedly better than those of the industry standard cetrimonium chloride.

Combinations of various conditioning agents with alkyl-guanidinium compounds were tested in simple hair rinses. The formulations can be taken from the following table.

TABLE 9
Test formulation of a simple hair rinse for the combination of
various conditioning agents with the alkylguanidinium
compounds according to the present invention:

| | Hair rinse Type D |
|---|---|
| | (according to the invention) |
| Alkylguanidinium compound | 1.0 |
| [% by weight] | |
| Conditioning agent of the prior art | 1.0 |
| [% by weight] | |
| Ceteareth-25 | 0.5 |
| [% by weight] | |
| Cetyl alcohol | 2.0 |
| [% by weight] | |
| HCl | to pH = 5 |
| Water | to 100 |
| [% by weight] | |

In the following table (Table 10), the results of the sensory assessment of the treatment of the strands of hair were carried out as described above with compounds according to the present invention in combination with conventional conditioning agents or comparison examples (formulations according to Tables 7 and 9) is summarized.

TABLE 10
Results of the sensory assessment after treatment of strands of hair with
formulations according to Tables 7 and 9 for the comparison of the action
in combination of the compounds according to the present invention with
conditioning agents of the prior art:

| | Compounds used | | | | |
|---|---|---|---|---|---|
| | Alkylguanidinium | | | | |
| | compound | | | | |
| | according to | | Sensory assessments | | |
| | formula (I) used | | | | Overall |
| Test | where R¹ = H | | | | assessment |
| formulation | present as the acetic | Conditioning agent of | Combability | Handle | (mean |
| Hair rinse type | acid salt | the prior art | wet | dry | value) |
| HSP A | HSP A where R² = C₂₂ | — | 4.2 | 4.5 | 4.5 |
| (according to | | | | | |
| the invention) | | | | | |
| HSP D | HSP A where R² = C₂₂ | Cetrimonium chloride | 4.4 | 4.6 | 4.6 |
| (according to | | | | | |
| the invention) | | | | | |
| HSP D | HSP A where R² = C₂₂ | Behentrimonium | 4.9 | 4.8 | 4.8 |
| (according to | | chloride | | | |
| the invention) | | | | | |
| HSP D | HSP A where R² = C₂₂ | Distearyldimonium | 4.1 | 4.4 | 4.3 |
| (according to | | chloride | | | |
| the invention) | | | | | |
| HSP D | HSP A where R² = C₂₂ | Quaternium-80 (ABIL | 5.0 | 5.0 | 5.0 |
| (according to | | Quat 3474, Degussa | | | |
| the invention) | | Care Specialties) | | | |
| HSP B | — | Cetrimonium chloride | 3.7 | 4.2 | 4.0 |
| (comparison | | | | | |
| recipe) | | | | | |
| HSP B | — | Behentrimonium | 4.8 | 4.3 | 4.6 |
| (comparison | | chloride | | | |
| recipe) | | | | | |
| HSP B | — | Distearyldimonium | 3.6 | 3.9 | 4.0 |
| (comparison | | chloride | | | |
| recipe) | | | | | |
| HSP B | — | Quaternium-80 (ABIL | 4.5 | 4.0 | 4.2 |
| (comparison | | Quat 3474, Degussa | | | |
| recipe) | | Care Specialties) | | | |
| HSP C | — | — | 1.7 | 3.0 | 2.6 |
| (comparison | | | | | |
| recipe; control | | | | | |
| basis) | | | | | |

The examples show that in the case of the use of the alkylguaridinium derivate according to the present invention in hair rinses a further improvement can be achieved if; in addition to the alkylguaridinium compound, further conditioning agents of the prior art were used. Such hair rinses containing combinations of conditioning agents of the prior art and alkylguaridinium derivatives were assessed to be better in the conditioner action than hair rinses which contain the respective individual ingredients.

While the present invention has been particularly shown and described with respect to preferred embodiments thereof it will be understood by those skilled in the art that the foregoing and other changes in forms and details may be made without departing from the scope and spirit of the present invention. It is therefore intended that the present invention not be limited to the exact forms and details described and illustrated, but fall within the scope of the appended claims.

What we claim is:

1. A hair treatment composition or hair after-treatment composition comprising, as a conditioning agent, 0.05 to 10.0% by weight at least one alkylguanidine compound of general formulae (I), (II) or a combination of (I) and (II) 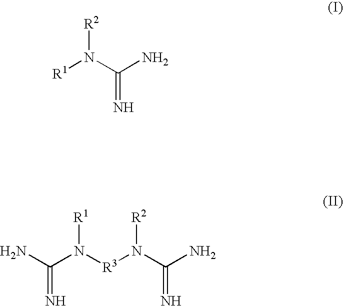
and/or its salts or hydrates, in which
R¹, R² a) independently of one another are H, a hydrocarbon radical, hydroxy-alkyl, alkyloxy, carboxyalkyl radicals, having 2 to 30 C atoms, or
b) alicyclic, heterocyclic compounds, which can have a ring size of 3 to 10 atoms, or aromatic compounds which can carry further, saturated or unsaturated hydrocarbon substituents having 1 to 30 C atoms, or
c) alkylamidoalkylene or alkyl ester alkylene radicals, which can further contain structural elements mentioned under a) and b), with the proviso that in compounds as shown in formula (I) where R¹=alkylamido-alkylene, R² is not hydrogen,
and the structural elements a), b), c) can be combined among one another and with one another,
R³ is alkylene, a hydrocarbon radical having 1 to 30 C atoms, or alicyclic or heterocyclic components, which is saturated, unsaturated or aromatic, having a ring size of 3 to 10 atoms, or in which R¹ and R² in the element —N(R¹)—R³—(R²)N— can form a 5- to 8-membered ring.

2. The hair treatment composition or hair after-treatment composition as claimed in claim 1, wherein at least one of the radicals R¹, R² is a C₈- or a C₁₆₋₂₂-alkyl radical.

3. The hair treatment composition or hair after-treatment composition as claimed in claim 1, wherein the radicals R¹, R² independently of one another are C₄-, C₁₀-, C₁₈-, coconut or tallow fatty acid or isotridecylalkyl radicals.

4. The hair treatment composition or hair after-treatment composition as claimed in claim 1, wherein the radicals R¹, R² are identical or different alkylamidoalkylene or alkyl ester alkylene radicals.

5. The hair treatment composition or hair after-treatment composition as claimed in claim 1, further comprising 0 to 10% by weight of one or more emulsifiers, 0 to 10% by weight of one or more consistency-imparting agents, 0 to 10% by weight of one or more surfactants, 0 to 20% by weight of one or more cosmetic oils or emollients, and customary excipients and additives in customary concentrations.

6. The hair treatment composition or hair after-treatment composition as claimed in claim 1, further comprising one or more hair cosmetic active ingredients selected from the group consisting of the protein hydrolyzates of vegetable or animal origin based on keratin, collagen, elastin, wheat, rice, soybeans, milk, silk corn or vitamins, panthenol, pyrrolidonecarboxylic acid, bisabolol, plant extracts, creatine, and ceramides.

7. A hair treatment composition or hair after-treatment composition for the cosmetic treatment of the hair and of the scalp against dandruff comprising at least one compound of general formulae (I), (II) or a mixture thereof 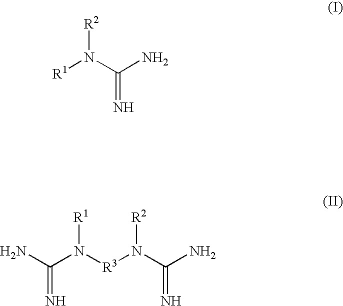
and/or their salts or hydrates, in which
R¹, R² a) independently of one another are H, a hydrocarbon radical, hydroxy-alkyl, alkyloxy, carboxyalkw radicals, having 2 to 30 C atoms, or
b) alicyclic, heterocyclic compounds, which can have a ring size of 3 to 10 atoms, or aromatic compounds which can carry further, saturated or unsaturated hydrocarbon substituents having 1 to 30 C atoms, or
c) alkylanidoalkylene or alkyl ester alkylene radicals, which can further contain structural elements mentioned under a) and b), with the proviso that in compounds as shown in formula (I) where R¹=alkylamido-alkylene, R² is not hydrogen,
and the strutural elements a), b), c) can be combined among one another and with one another,
R³ is alkylene, a hydrocarbon radical having 1 to 30 C atoms, or alicyclic or heterocyclic components, which is saturated, unsaturated or aromatic, having a ring size of 3 to 10 atoms, or in which R¹ and R² in the element —N(R¹)—R³—(R²)N— can form a 5- to 8-membered ring.

8. A cosmetic formulation for the treatment of hair comprising an alkylguanidine compound of claim 7 which is free of fatty alcohols.

9. A hair shampoo, hair conditioning compositions or leave-in formulations comprising at least one alkylguanidine compound of claim 7.

10. A method of improving the soft feel and for the antistatic finishing of keratinic fibers comprising a step of applying an alkylguanidine compound of claim 1 to keratinic fibers.
